(19) United States
(12) Patent Application Publication
(10) Pub. No.: US 2005/0175699 A1
(43) Pub. Date: Aug. 11, 2005

(54) Multilamellar system for the administration of active agents by means of ingestion

(76) Inventor: Marie Elisabeth Cuine-Backert, St Fargeau-Ponthierry (FR)

Correspondence Address: Marie Elisabeth Cuine-Backert, 41 bis Allee de la Faisanderie, St Fargeau-Ponthierry, 77310, FR

(21) Appl. No.: 10/512,175

(22) PCT Filed: Apr. 29, 2003
(86) PCT No.: PCT/FR03/01353

(30) Foreign Application Priority Data
May 15, 2002 (FR) 02/05944

(51) Int. Cl. A61K 9/24

(52) U.S. Cl. 424/472

(57) ABSTRACT

Multilamellar device for the administration of active substances by ingestion. It is disclosed a device with a support film (2)(15) on which a preparation said matrix layer (12 to 14) constituted, of active(s), liquid or solid ingredients giving jellified preparations, is coated. It is possible to superimpose several matrix layers (12)(13)(14), with or without active, or superimpose different basic systems, giving superimposition of support films with identical or different formulations (2)(15) and matrix layers with identical or different formulations (12)(13)(14). The devices are cut in small units that can be used under various presentations. This multilamellar device gives a lot of possibilities for programming the release of actives used particularly to prepare new medicinal products.

The invention discloses a multilamellar device for the administration of actives substances (in particular medicinal products) by ingestion per oral route.

Numerous devices for administration of medicinal products through oral route have been described. There are liquids preparations (syrups, drops, solutions . . . ) or unit solids doses (tablets, hard capsules, soft capsules . . . ). The chosen device depends of medicine type (Active Pharmaceutical Ingredient, API after in the text) to administer and intended release profile for active ingredient (fast, slow . . . ).

Today there is not one device able by itself to manage the large variety of actives substances to administer. Liquids API or API formulated as a solution, cannot be usually administered as tablets. API and/or ingredients for hydrophilic formulations, presented as liquids are not very compatible with hard or soft capsules. Moreover new API developed in pharmacy needs more and more the use of sophisticated delivery programs and/or sophisticated formulations in order to give: an absorption improvement, a targeting in the time and/or in the space, amorphous API stabilization. These constraints oblige to more or less complex adaptations of today administration devices.

The multilamellar device disclosed in this invention, independently of his novelty to administer medicines per oral ingestion, gives the possibility to avoid difficulties with today's oral forms, through a unique formulation device with multiple possibilities, using a simple and efficient industrial process.

The invention describes a device which gives an answer to new needs of sophisticated delivery programs of API and avoids difficulties enunciated here before, because this device gives possibility:
to administer per oral route, API of very different nature: powders in solution, powders in suspensions, hydrophilic or lipophilic liquids for example,
to administer in an interesting way, API in great proportion, in particular liquids, or API in low quantities, or very toxics API.
surprisingly to administer with an unique system, one or more API identical or different,
to make formulations with incompatible API,
to easily adapt dosage,
to combine in an interesting way different release profiles for identical or different API, giving the possibility to get the needed and sophisticated liberation profile for each API contained in the system and trough a unique system.

This new unique device is constituted of a basic multilamellar device use as it is, advantageously as a flat system, or, in a new and surprising way by superimposing one or multiple basic, identical or different multilamellar devices.

The basic multilamellar device, purpose of this invention is constituted of, a homogeneous polymeric support film, not containing API, on which a homogeneous preparation (constant concentration in API on the entire surface), containing one or more API, is coated, with a constant thickness on the entire surface (for example by laminating) in order to constitute the matrix layer.

This device, can according to particular ways of preparation, be constituted: of one or more liquid or solid API, of ingredients or additives with variable physicochemical properties: aqueous liquids, oily liquids and powders.

The basic multilamellar device disclosed in the invention, in addition to the matrix layer containing one or more API, can be constituted of several superimposed matrix layers, each one being homogeneous with a constant thickness on the entire surface for one given layer, coated successively, containing or no for each of them API.

The basic multilamellar device can be cut in units and used as it is, advantageously in a flat shape, in order to give multiple possibilities of release profiles (including for example delayed, and/or extended, and/or pulsatile release).

One and/or several basic multilamellar devices, identical or with different formulations, can be superimposed, in order to give multiple possible release profiles.

The support film is a polymeric film, soluble or insoluble in digestive liquids, homogeneous, with a constant thickness on the entire surface.

In order to produce support films, in addition to ingredients and/or additives (be. plasticizers), polymers or copolymers (obtained by reaction of listed polymers in various proportion), listed in a non-limitative way hereafter, can be used (in dispersion in appropriate solvents), alone or in mixture in order to produce films more or less soluble or insoluble in digestive liquids.

The different ingredients, additives, polymers or copolymers are homogeneously mixed, without adding API, and after with classical technologies for films manufacture, cold or hot process, films are produced with a constant thickness on the entire surface.

The drying of the films is done by heating and/or with infrared systems and/or with microwaves systems.

In order to produce the matrix layer (3) polymers or copolymers used for support films (2) can be used (in dispersion in appropriate solvents) as well as pharmaceutical ingredients, absorption promoters agents, emulsifying agents, texture agents, flavoring agents or sweeteners, adhesives. Ingredients are listed hereafter, the list being not limitative.

Ingredients and manufacturing processes used for matrix layer give jellified preparations in form of: solution, suspension, emulsion, pre-emulsion, micro-emulsion, pre-micro-emulsion. After mixing of different constituents, coating is performed in order to obtain matrix layers (3) with a constant thickness throughout the entire surface for a given matrix layer. Cold or hot coating is done. If needed the drying is performed by heating (preferably between 20° C. and 100° C.) and/or infrared lamps and/or using microwaves.

Advantageously ingredients permutation and combination in the matrix layer (liquid, semi-solid or solid API, ingredients, additives, with different physicochemical properties: aqueous, oily, powdered) give the possibility to realize a large number of formulations with various and programmed release profiles, adapted to the API (<1 hour), and/or prolonged release over a 24 hours period, and/or delayed release (with 1 to 12 hours lag time), and/or pulsatile delivery (successive releases with a lag time in between varying from 1 to 12 hours). These possibilities are increased if several matrix layers are superimposed.

A characteristic of the invention is to authorize the coating in the same time (same coating phase), in parallel lines or following a predetermined geometrical figure, of one or several matrix layer preparations (4), (5), (6), (7), (8), (9), (10), (11) in order to constitute a layer containing different formulations.

This disposal as shown on FIGS. 2 and 3, give in addition the possibility to increase the number of possible release programs, and also to formulate incompatible API.

Surprisingly and advantageously possibilities of formulations and delivery programs are increased by the fact that from a key characteristic of the invention it is possible to superimpose one or several basic multilamellar devices, identical or different, giving at the end superimposition of support films with identical or different formulations (2), (15) and matrix layers with identical or different formulations (12), (13), (14), as illustrated on FIG. 4.

Advantageously and surprisingly, the ingenious choice of support films and matrix layers formulations in a device with or without superimposition of one or several basic devices, give the possibility to obtain, simply using the device as it is, in a flat shape, after cutting it in units, in agreement with an other characteristic of the invention, multiple and sophisticated release programs adapted to each API, without the necessity to roll or fold up the devices.

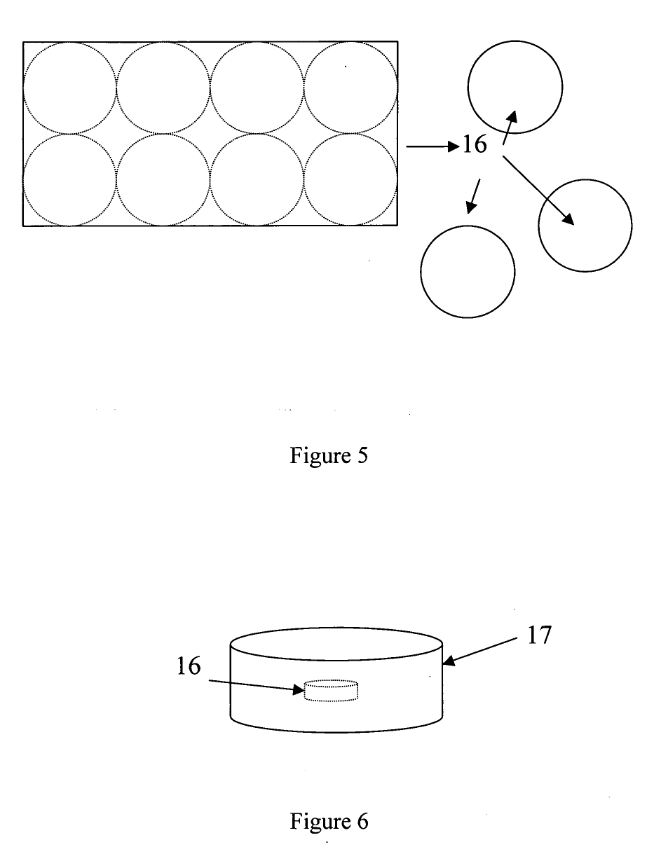

By cutting devices in order to obtain small flat units (16), it is possible to produce, as illustration of the invention, devices with a disk shape (FIG. 5), cubic shape, parallelepiped shape.

Before or after cutting, devices can have been submitted to an operation like: folding up, rolling . . . without to be limited by this list of operations, in order to obtain shapes for the devices adapted to the need.

Folding up and rolling can necessitate adhesives on or in the superior matrix layer or on predetermined part of the support film. In agreement with the invention it is also possible to seal support films with state of the art techniques, by example by heating.

In agreement with one characteristic of the invention, the units obtained using described processes in one of the previous characteristics, can be package are they are in a primary pack like: small bag, blister, or bottle . . .

They can equally be filled in a capsule, a tablet (17) (FIG. 6), or in a soft capsule, or been coated (films, “waffles” . . . ), with the necessary ingredients and/or additives.

Advantageously one or several units or devices (with identical or different formulations) can also be associated with one or several others pharmaceutical forms and/or powdered, liquid or semi-solid ingredients (FIG. 8).

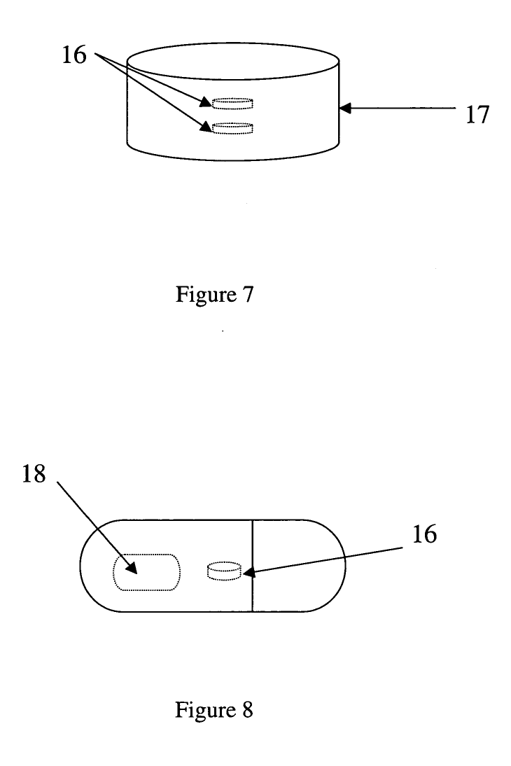

Several units (16) different or with the same formulation can be put with the necessary ingredients in one hard capsule or soft capsule or tablet (17) (FIG. 7), by example. In addition last two possibilities can be associated.

In this manner the invention permits to answer to the new needs in Pharmaceutical Sciences, that means, to obtain a sophisticated combination of release profiles for identical or different API and to formulate a large variety of API.

The dosage in API can be easily changed by simply adapting area of the device by cutting it when preparing the units or by modifying the number of basic devices superimposed or by regrouping several identical or different devices in an other pharmaceutical form, like by example a tablet.

For the preparation of support films (2) polymers or copolymers (obtained by reaction of listed polymers in various proportions) listed hereafter can be used (in dispersion in appropriate solvents), the list being non limitative: polyvinyl acetate, acrylics and derivatives, polyvinyl alcohol, cellulose and derivatives (cellulose acetophtalate, sodium carboxymethylcellulose, ethylcellulose, hydroxyethylcellulose, hydroxypropylcellulose, methylcellulose, methylhydroxyethylcellulose, methylhydroxypropylcellulose), polyvinyl chloride, ethylenevinylacetate (EVA), fluorocarbones, glycerol, polyesters, polyolefins, polyethers, polyethylenes, polyamids, polyethylene glycols, polyimids, natural polymers (gelatin, acacia gum, alginates, starchs and derivatives, tragacanth gum, carraghenates, chitosans), polysiloxans, polypropylene, polyurethanes, polyvinylpyrrolidon and derivatives like copolymers of vinyl pyrrolidon and vinyl acetate, propylene glycol, copolymer of poly(methyl vinyl ether/maleic anhydrid).

In order to realize matrix layer (3) polymers or copolymers used for support films (2) can be used (in dispersion in appropriate solvents) as well as absorption promoters, emulsifying agents, texture agents, flavoring agents or sweeteners, adhesives ; by example ingredients listed hereafter, the list being non limitative: fatty acids and derivatives, fatty alcohols and derivatives, saturated or unsaturated polyglycolisated glycerids, Nmethyl-2-pyrrolidon, emulsifying agents (like sorbitan esters and polyoxyethylenated sorbitan esters, oleates, animal or vegetal lecithin's, polyoxyethylenated ricin oil, sucro esters, esters of fatty acids and polyethylene glycol, esters of fatty acids and glycerol, esters of fatty acids and propylene glycol, bile acides and salts), diethylene glycol monoethyl ether, dimethylisosorbide, terpens, phospholipids, polycarbophils (like by example products commercialized as “CARBOPOL” by BFGoodrich), vegetal or mineral oils, carbohydrates, cyclodextrins and derivatives, silice and derivatives, acids et bases for pH adjustment.

The device in the disclosed invention can be use for classes of API listed hereafter, the list being not limitative or extensive: inflammatory agents and analgesics, like by example corticoids, indomethacin, ibuprofene, diclofenac, tenoxicam or piroxicam, antiseptics, vasodilatators agents like nitroglycerin or isosorbide dinitrate, antiasthmatic agents, anti-bacterial agents, antibiotics, cardiotonics, anesthesics like lidocaine, anti-angor agents, anti-arhythmics, anti-hypertensives, anti-aggregants agents, antitussives and expectorants like codein, antihistaminics like chlorphenamin, dopaminergics agonists, sleep regulators like melatonine, hemostase promotors, hormones, anti-tumor agents, anti headache agents, anti-parkinson, memory stimulants, antidepressives, anxiolytics, hypnotics, antidiabetics, anti-obesity agents, antifongiques, antiviral agents, anti-ulcer drugs, anti-diarrhea.

Annexed DRAWINGS illustrate the invention:

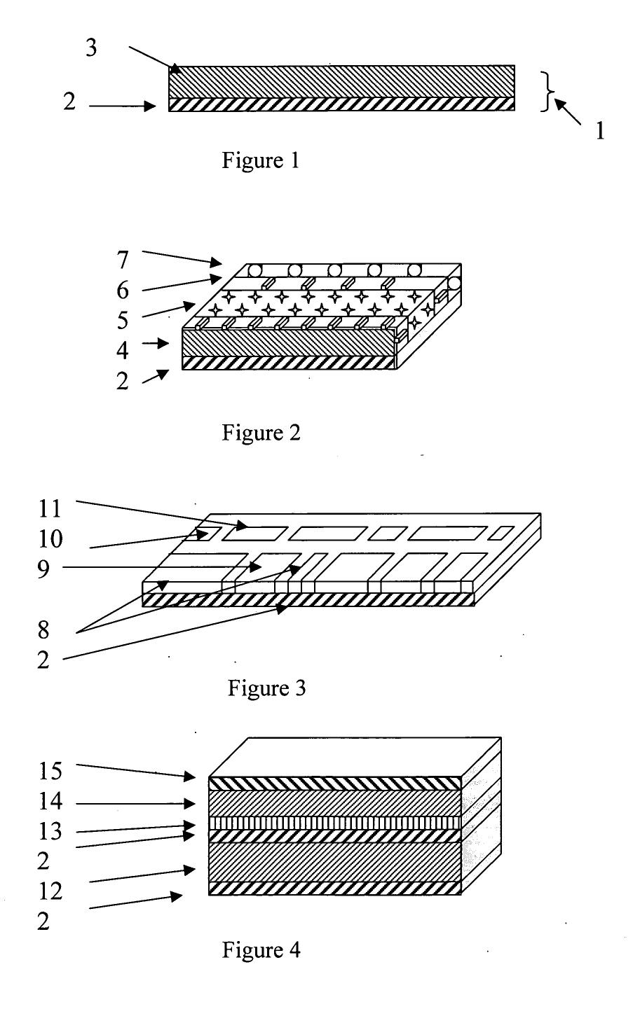

FIG. 1 represents a section of a basic multilamellar device (1) by coating a matrix layer (3) on a support film (2).

FIGS. 2 et 3 show an other characteristic of the device, that means a parallel coating (FIG. 2) or with a defined geometrical disposition (FIG. 3) of different preparations of matrix layers (4), (5), (6), (7), (8), (9), (10), (11).

FIG. 4 illustrates un other characteristic of the invention, that means the superimposition of different devices, conducting to the superimposition of support films (2), (15) with identical or different formulations and matrix layers with identical or different formulations (12), (13), (14).

FIG. 5 illustrates an other characteristic of the invention, that means the possibility to cut the devices in units (16).

FIGS. 6 à 8 show an other characteristic of the invention, that means the fact to package one or several units (16), as they are in a primary pack, by example a blister pack, or by filling one or several units (16) in an other pharmaceutical form, by example in a tablet (FIGS. 6 et 7), alone or associated with others pharmaceutical forms (FIG. 8).

The following EXAMPLES illustrate the invention without limiting them in any manner.

### EXAMPLE 1

Step 1: dissolve 10 g of nifedipin ins 200 g of polyoxyethylene glycol 400 and make gel by adding 15 g of AEROSIL 200.

Step 2: coat the mix prepared at step 1 on a support film (2) constituted of hydoxypropylcellulose, hydroxypropylmethylcellulose and polyethylene glycol 400 in the following proportions 60/30/10. The mass coated (3) is 15 mg/cm² of support film (2).

Step 3 roll and fold up the device in order it contains 10 mg of nifedipin per unit and fill the units in size 0 capsules with 50 mg of lactose and 0.5 mg of magnesium stearate.

### EXEMPLE 2

Step 1: dissolve 5 g of indometacin and 0.5 g of Carbomer 1342 (supplied by BFGoodrich) in 100 g of oleic acid, and make gel by adding 8 g of AEROSIL R972.

Step 2: coat the mix prepared in step 1 on a support film (2) constituted of hydoxypropylcellulose, hydroxypropylmethylcellulose and polythylene glycol 400 in prroportions 60/30/10. The mass coated (3) is 15 mg/cm² of support film (2).

Step 3: roll and fold up the coated device in order it contains 20 mg of indomethacin per unit and fill in size 0 capsules with 50 mg of lactose and 0.5 mg of magnesium stearate.

### EXAMPLE 3

Step 1: disperse 20 g of nifedipin in 200 g of polyoxyethylene glycol 400 and make gel by adding 15 g of AEROSIL 200.

Step 2: coat the mix prepared at step 1 on a support film (2) constituted of ethylcellulose and polyethylene glycol 400 in proportions 90/10. The mass coated (3) is 15 mg/cm² of support film (2).

Step 3: roll and fold up the device in order to have 20 mg of nifedipin per unit and fill the units in size 0 capsules with 50 mg of lactose and 0.5 mg of magnesium stearate.

### EXAMPLE 4

Step 1: dissolve 2 g of lysine acetylsalicylate (expressed in acid equivalent) in a solution constituted of 12 g of PVP K29/32, 1 g of Carbopol 974, 0.15 g of glycerol, 20 ml of water and 13 ml of ethanol.

Step 2: coat the mix prepared in step 1 on a support film (2) constituted of PVP K29/32 with 6% (m/m) of Carbopol 974 and 1% (m/m) of glycerol. The mass coated is (3) 30 mg/cm² of support film (2). The support film is, prior to be coated with the mix prepared at step 1, coated with an adhesive Durotak 387-2516.

Step 3: dry under hot air at 50° C. until to obtain a dry film and cut the device in pieces of 2×2 cm.

Step 4: study in vitro dissolution of acetylsalicylic acid in apparatus n° 1 of European Pharmacopea, in water at 37° C., with U detection at 265 nm. We observe a kinetic of dissolution of order 1 with 50% dissolution of API at 30 minutes and 100% in 2 hours.

### EXAMPLE 5

Step 1: dissolve 2 g of lysine acetylsalicylate (expressed in acid equivalent) in a solution constituted of 12 g of PVP K29/32, 1 g of Carbopol 974, 0.15 g of glycerol, 20 ml of water and 13 ml of ethanol.

Step 2: coat the mix prepared at step 1 on a support film (2) constituted of ethylcellulose, hydroxyethylcellulose and glycerol in proportions 45/45/10. The mass coated (3) is 30 mg/cm² of support film (2). The support film is, prior to be coated with the mix prepared at step 1, coated with an adhesive Durotak 387-2516.

Step 3: dry under hot air at 50° C. until to obtain a dry film and cut the device in pieces of 2×2 cm.

Step 4: study in vitro dissolution of acetylsalicylic acid in apparatus n° 1 of European Pharmacopea, in water at 37° C., with UV detection at 265 nm. We observe a kinetic of dissolution of order 0 with 50% dissolution of API in 1 H 30 and 100% in 4 hours.

The multilamellar device described in this invention is particularly designed to formulate new medicines for pharmaceutical industry.

1. Multilamellar device (1) for administration of at least one active substance, particularly medicines, per oral route per ingestion, characterized by a polymeric support film (2), without active substance, with a constant thickness on the entire surface, soluble or insoluble in digestive liquids, constituted of polymers and/or co-polymers, on which at least one matrix layer (3) containing one or several active substances, in constant concentration on the entire surface, is coated with a constant thickness on the entire surface;
Such a device:
as it is in a flat shape after cutting in units,
or by superimposition of one or several basic devices (one support and one or several matrix layers), identical or different, giving superimposition of support films with identical or different formulations (2)(15), and matrix layers with identical or different formulations (12)(13)(14),
and/or associated with one or several others pharmaceutical forms,
and/or put in a hard capsule or a soft capsule or a tablet,
and/or coated,
giving a large variety of programmed release profiles of active (s):

2. The device according to claim 1 wherein a support film is constituted of ingredients and/or polymers and/or copolymers more or less soluble or insoluble in digestive liquids.

3. The device according to claim 1 wherein a support film can be coated on predetermined part with an adhesive.

4. The device according to claim 1 wherein a matrix layer is constituted of a jellified formulation in type of solution, suspension, emulsion, pre-emulsion, micro-emulsion, pre-micro-emulsion.

5. The device according to claim 1 wherein the matrix layer is constituted of ingredients and/or polymers and/or co-polymers giving a release profile specific to each active substance: immediate release (<1 hour), and/or prolonged release over 24 hours, and/or delayed release (with a lag time of 1 to 12 hours), and/or pulsatile delivery (successive release with a lag time in between of 1 to 12 hours).

6. The device according to claim 1 wherein the matrix layer can contain different formulations, by coating, in parallel lines (4), (5), (6), (7), or according a predetermined geometrical disposal (8), (9), (10), (11).

7. The device according to claim 1 wherein in addition to the matrix layer containing one or more actives, the device can be constituted of one or more matrix layer without active.

8. The device according to claim 1 wherein the matrix layer can contain one adhesive or be coated with an adhesive.

9. The device according to claim 1 wherein systems according to one of claims 1 to 8 can be superimposed, giving superimposition of support films of identical or different formulations (2), (15) and matrix layers of identical or different formulations (12), (13), (14).

10. The device according to claims 1 to 9 wherein the system obtained can be cut in units (16) with a flat form.

11. The device according to claims 1 to 9 wherein the device, with a flat shape, can be rolled and after cut to give cylindrical units.

12. The device according to claim 10 wherein the flat system can be fold up, before or after to be cut in units.

13. The device according to claims 10 to 12 wherein the units obtained can be coated.

14. The device according to claims 10 to 12 wherein the device can be put as it is in a primary packaging.

15. The device according to claims 10 to 12 wherein the device can be put in a tablet (17) with the necessary ingredients and/or additives.

16. The device according to claims 10 to 12 wherein the device can be put in a hard capsule, in a soft capsule, alone or with powdered, liquid or semi-solid-ingredients.

17. The device according to claims 10 to 12 wherein several devices with the same or different formulations can be put with the necessary ingredients in a hard capsule, in a soft capsule or a tablet (17).

18. The device according to claims 10 to 12 wherein one or several devices with the same or different formulations can be associated with one or several others pharmaceutical forms (18).

19. The device according to claims 10 to 12 wherein one or several devices with the same or different formulations can be associated with one or several others pharmaceutical forms (18) and put in a hard capsule, a tablet or a soft capsule with the necessary ingredients.
